(19) United States
(12) Patent Application Publication
(10) Pub. No.: US 2017/0319130 A1
(43) Pub. Date: Nov. 9, 2017

(54) SYSTEM, METHOD AND APPARATUS FOR DETERMINING HYDRATION LEVELS IN ANIMALS

(71) Applicants: Shawn Thomas, New York, NY (US); Jennifer Chi Xiong, Houston, TX (US); Daniel Huang, Charlestown, MA (US); Jason Tong-ho Suh, Edison, NJ (US)

(72) Inventors: Shawn Thomas, New York, NY (US); Jennifer Chi Xiong, Houston, TX (US); Daniel Huang, Charlestown, MA (US); Jason Tong-ho Suh, Edison, NJ (US)

(21) Appl. No.: 15/145,359

(22) Filed: May 3, 2016

(51) Int. Cl. A61B 5/00; A61B 10/00; A61B 5/1468; A61B 5/145; A61J 9/00

(52) U.S. Cl. CPC A61B 5/4875; A61J 9/00; A61B 10/0051; A61B 5/742; A61B 5/4836; A61B 5/0004; A61B 5/1468; A61B 5/14507; A61B 5/0022; A61B 5/4277

(57) ABSTRACT

A system, apparatus and method for saliva-conductance based hydration sensors and for a wireless network comprising hydration sensors and base stations. The hydration sensors include electrodes, a means of measuring saliva conductance, a processor, memory and wireless controllers. The hydration sensors output user hydration status based on the conductance of the user's saliva. Saliva conductance is correlated with ion concentration of saliva, which is a biomarker for dehydration. Thus, saliva conductance can be a proxy for dehydration. The hydration sensors may be used in active or hostile conditions, e.g., military use. Symptoms of dehydration are delayed: fatigue and impaired judgment can occur before a user realizes he/she is dehydrated. The hydration sensors indicate dehydration to a user, thus encouraging a user to hydrate and prevent dehydration-related injuries. Data from hydration sensors may be used in determining supply delivery logistics in hostile or inaccessible terrain.

### BACKGROUND

### Field of invention

Embodiments of the present disclosure relate generally to fluid conductance measurements and, more specifically, to determining hydration levels in animals.

### Description of Related Art

Many individuals suffer from dehydration, which could be largely prevented by maintaining proper hydration levels. Those in extreme environments are particularly prone to hydration-related issues due to heat, dangerous levels of physical activity, inability to judge the severity of potential dehydration, and lack of continuous water access. Crucially, the symptoms of dehydration are often delayed: fatigue and impaired judgment can occur well before an individual is able to prevent dehydration and associated symptoms. While dehydration can be treated by increased water consumption, extreme environment conditions make proper hydration status difficult to accurately and objectively monitor.

Publications disclose methods and devices that determine the hydration status of individuals through variegated means. Conventional methods, some of which are considered industry standards for measuring dehydration, include urine and blood chemical composition analysis, plasma osmolality, as well as body weight differential analysis due to water loss. These methods are impractical due to their invasiveness and interference with in-field activities, such as may be found in military use.

One such device uses ultrasonic sensing to measure hydration levels of tissue. Ultrasonic velocity may be used as an indicator of tissue hydration and thus overall hydration status. This is accomplished by positioning two ultrasonic transducers at a fixed distance away from each other across the tissue of interest. By measuring the time it takes for an ultrasonic pulse to travel the known distance, one can then calculate the ultrasonic velocity. However, such a device has not been proven to be sensitive enough to detect the minor changes in tissue hydration due to fluid loss. Also, the correlation between tissue hydration and overall hydration status is not well documented. Finally, such a device would not be suitable in field settings because the device would not be operational in vigorously active conditions, such as outdoors or in combat settings.

Another device for measuring hydration uses saliva flow rate due to capillary action. It is known that the rate of flow into a water-permeable material is correlated to saliva concentration. However, this device includes several drawbacks to in-field use; one such drawback is that it takes several minutes to take a measurement. This device includes a timing apparatus that keeps track of the time elapsed during measurement. Thus, a saliva-flow rate device is impractical to use in outdoor or extreme environments.

Another method for determining hydration levels includes analyzing sodium concentration in saliva. This method includes mixing saliva with a second solution to make a third solution, followed by dipping a chemically-treated piece of paper into the third solution. The user must then wait for the chemicals deposited on the paper to change in color, based on absorption of the third solution of the paper and mixture with the chemicals deposited on the paper. Clearly, such a method is cumbersome, time-consuming and is not appropriate for in-field, real-time hydration measurements.

An additional method of hydration sensing includes determining saliva osmolarity using a bench top device known as a freezing-point depression osmometer. Similar to methods and devices listed above, this method does not provide for a portable means of hydration level testing. Rather, this method is slow and cumbersome, in part due to requirements that the user perform chemical analysis using a non-portable osmometer.

As the foregoing illustrates, what is needed in the art is a simple, portable, accurate and fast method of determining hydration levels in humans and other animals.

### SUMMARY

Disclosed herein is a system including: a wireless network comprising hydration sensors and base stations. The hydration sensors include two or more electrodes, a means of measuring saliva conductance, a processor, memory and wireless controllers. The hydration sensors can output hydration status data of the users. The base station coordinates hydration status data between each of the hydration sensors.

Embodiments according to the present disclosure may use saliva conductance as a biomarker for dehydration. In one embodiment, a user may deposit a saliva sample into a collector which has exposed electrodes at the base of the collector. In another embodiment, the user may simply place the hydration sensor into the mouth and effectuate electrode contact with the user's saliva.

Once the saliva makes contact with these electrodes, embodiments according to the present disclosure may determine conductance using the electrodes. Then, embodiments according to the present disclosure may process the conductance and determine hydration status of the user. Finally, embodiments according to the present disclosure may provide an output indicating hydration status.

Embodiments according to the present disclosure may provide for a lightweight, portable and nonintrusive form factor that causes minimal interference with field activity. Such field activity may include military combat conditions or vigorously active use generally. Embodiments according to the present disclosure may also provide for simple, straightforward use and real-time measurement capability to help ensure compliance among users. Finally, embodiments according to the present disclosure may allow for ease of integration into existing equipment, such as installation onto catheters included with popular hydration sport backpacks, and other portable hydration systems.

Indeed, numerous, variegated uses of embodiments according to the present disclosure are possible, including but not limited to: (1) militaries could monitor hydration levels of soldiers in order to determine logistics (e.g., where to deliver more water/supplies); (2) hospitals and researchers could include hydration sensing as part of a standard list of vital sign checkups, to gather clinical trial data as well as determine the efficacy of being well-hydrated or under-hydrated on various treatments; (3) pharmaceutical companies could gather clinical trial data on how hydration affects the efficacy and side effects of drug therapy; (4) athletics could determine hydration levels during gameplay and thus control water intake vs. urination, thus influencing on- vs. off-court time; (5) outdoor enthusiasts could figure out how much water they are using and how much water they need to use and share their data on social media; (6) wearable device manufactures could encourage more water usage, share data on social media, and incorporate hydration sensors according to embodiments of the present disclosure as part of the wearable device; (7) pilots and astronauts could monitor their hydration status as part of vital sign tracking and (8) doctors could recommend additional hydration therapy to their patients who have a hydration sensor according to embodiments of the present disclosure.

The construction and method of operation of the invention, however, together with additional objectives and advantages thereof will be best understood from the following description of specific embodiments when read in connection with the accompanying drawings.

### BRIEF DESCRIPTION OF THE DRAWINGS

FIG. 1A illustrates an exploded view of a dehydration sensor, according to one embodiment of the present disclosure;

FIG. 1B illustrates an exploded view of a dehydration sensor, according to one embodiment of the present disclosure;

FIG. 2 illustrates a circuit diagram, according to one embodiment of the present disclosure;

FIG. 3 illustrates a cloud computing environment, according to one embodiment of the present disclosure;

FIG. 4 illustrates a flow diagram of method steps for determining hydration levels, according to one embodiment of the present disclosure;

FIG. 5A illustrates a graph indicating saliva conductance vs. saliva osmotic concentration in human subjects, according to one embodiment of the present disclosure;

FIG. 5B illustrates a graph indicating saliva conductance vs. saliva osmotic concentration in human subjects, according to one embodiment of the present disclosure;

FIG. 5C illustrates a graph indicating saliva conductance vs. saliva osmotic concentration in human subjects, according to one embodiment of the present disclosure;

FIG. 5D illustrates a graph indicating saliva conductance vs. saliva osmotic concentration in human subjects, according to one embodiment of the present disclosure;

FIG. 6 illustrates a hydration sensor integrated into military equipment, according to one embodiment of the present disclosure;

FIG. 7A illustrates an integrated hydration sensor and hydration catheter, according to embodiments according to the present disclosure;

FIG. 7B illustrates an integrated hydration sensor and hydration catheter, according to embodiments according to the present disclosure;

FIG. 8A illustrates an integrated hydration sensor and hydration catheter, according to embodiments according to the present disclosure;

FIG. 8B illustrates an integrated hydration sensor and hydration catheter, according to embodiments according to the present disclosure;

FIG. 9 illustrates an integrated hydration sensor and hydration catheter and reservoir, according to embodiments according to the present disclosure; and,

FIG. 10 illustrates a hydration sensor attached to a hydration bottle system, according to one embodiment of the present disclosure.

### DESCRIPTION

### Generality of Invention

This application should be read in the most general possible form. This includes, without limitation, the following:

References to specific techniques include alternative and more general techniques, especially when discussing aspects of the invention, or how the embodiment might be made or used.

References to “preferred” techniques generally mean that the inventor contemplates using those techniques, and thinks they are best for the intended application. This does not exclude other techniques for the invention, and does not mean that those techniques are necessarily essential or would be preferred in all circumstances.

References to contemplated causes and effects for some implementations do not preclude other causes or effects that might occur in other implementations.

References to reasons for using particular techniques do not preclude other reasons or techniques, even if completely contrary, where circumstances would indicate that the stated reasons or techniques are not as applicable.

Furthermore, the invention is in no way limited to the specifics of any particular embodiments and examples disclosed herein. Many other variations are possible which remain within the content, scope and spirit of the invention, and these variations would become clear to those skilled in the art after perusal of this application.

### DETAILED DESCRIPTION

Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

References in the specification to “one embodiment”, “an embodiment”, “an example embodiment”, etc., indicate that the embodiment described may include a particular feature, structure or characteristic, but every embodiment may not necessarily include the particular feature, structure or characteristic. Moreover, such phrases are not necessarily referring to the same embodiment. Further, when a particular feature, structure or characteristic is described in connection with an embodiment, it is submitted that it is within the knowledge of one of ordinary skill in the art to effect such feature, structure or characteristic in connection with other embodiments whether or not explicitly described. Parts of the description are presented using terminology commonly employed by those of ordinary skill in the art to convey the substance of their work to others of ordinary skill in the art.

### System Elements

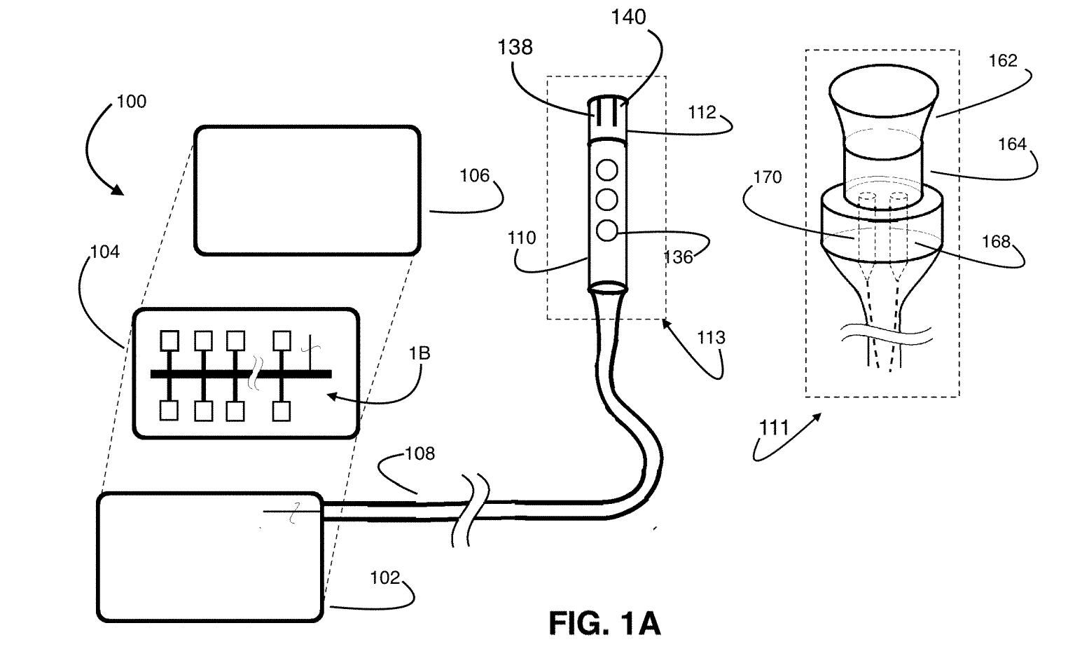

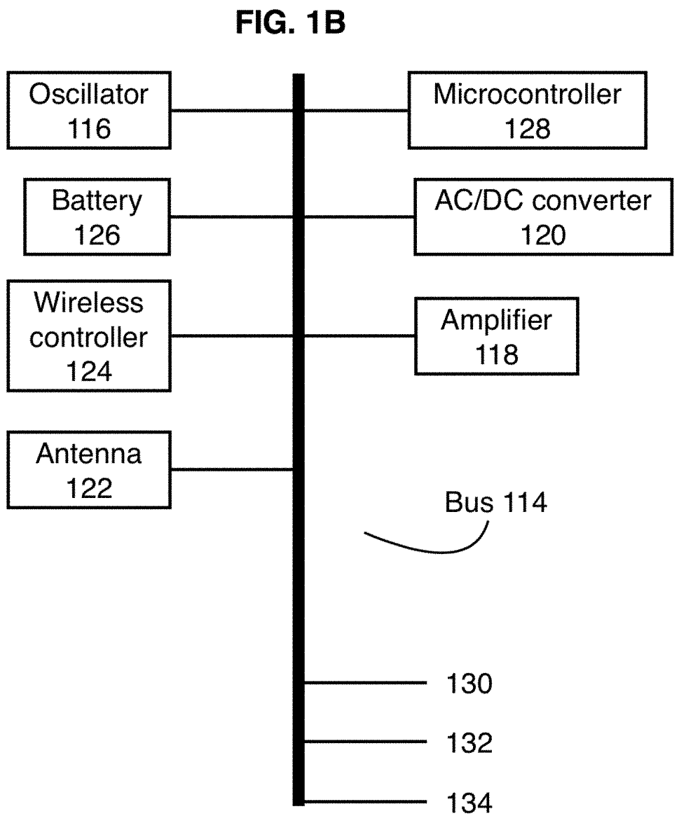

FIG. 1

FIGS. 1A and 1B illustrate an exploded view of a dehydration sensor, according to one embodiment of the present disclosure. As shown, the hydration sensor 100 includes a housing 102, a printed circuit board (PCB) 104, a cover plate 106, a cable 108, a stem 110, and a saliva conductance tester 112. By way of example and not limitation, saliva conductance tester 112 may take the form of mouthpiece 113, or saliva collector cap 111. In some embodiments, saliva conductance tester 112 may be disconnected and swapped with other types of saliva conductance testers 112 mentioned herein.

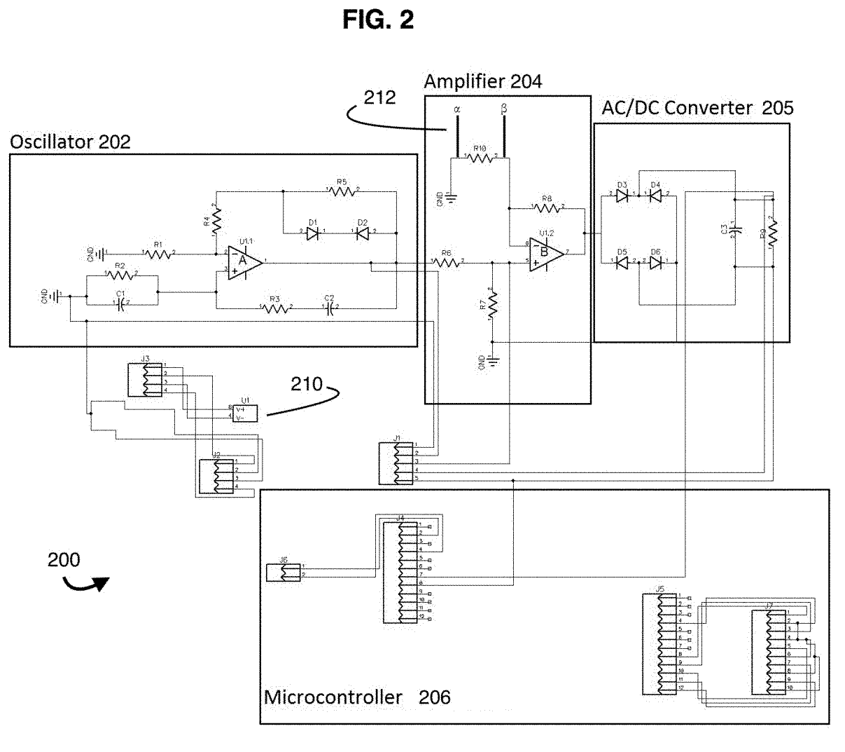

PCB 104 may be contained within housing 102, and beneath cover plate 106, which serve to protect PCB 104, among other functions. The circuitry in PCB is shown in detail in FIG. 1B. PCB 104 may include but is not limited to the following components connected to a bus 114: oscillator 116, differential amplifier 118, AC/DC converter 120, antenna 122, bluetooth circuit 124, battery 126, microcontroller 128. Bus 114 may also be connected to but not limited to the following components: positive electrical lead 130, negative electrical lead 132 and optionally, data leads 134. Data leads 134 may contain one or more distinct electrical connections. The interactive functionality of many of the elements comprising PCB 104 will be discussed in the detailed description for FIG. 2. (41) In some embodiments, housing 102 and/or cover plate 106 may be coupled to cable 108. In further embodiments, PCB 104 may be electrically connected to cable 108. By way of example and not limitation, PCB 104 may be electrically connected to cable 108 through one or more of the following components: positive electrical lead 130, negative electrical lead 132 and optionally, data leads 134.

In some embodiments, cable 108 may be coupled to stem 110 and saliva conductance tester 112. In further embodiments, stem 110 and/or saliva conductance tester 112 are electrically connected to PCB 104 through one or more of the following components: positive electrode 130, negative electrode 132 and optionally, data leads 134.

Stem 110 may have LEDs 136. In some embodiments, LEDs 136 may serve to indicate to the user levels of hydration, power on/off status, battery charge and other functions. Alternatively, LEDs 136 may be disposed on housing or cover plate 102 or 106 (not illustrated).

Saliva conductance tester 112 may be attached to positive electrode 138 and negative electrode 140. In some embodiments, positive electrode 138 and negative electrode 140 serve to test the conductance of saliva in proximity to saliva conductance tester 112. In some embodiments, positive electrode 138 and negative electrode 140 and other electrodes or saliva conductance measurement means described herein may be made of gold, platinum, copper, conductive polymer, carbon or the like. In one embodiment, mouthpiece 113 may be engulfed in saliva such that the saliva makes contact with both electrodes 138 and 140 such that an electrical current may flow through the saliva, thus allowing conductance to be determined. In other embodiments, both electrodes 138 and 140 may be structurally sound such that both electrodes 138 and 140 are resistant to crushing forces (e.g., biting).

As mentioned above, saliva conductance tester 112 may take the form of saliva collector cap 111. Saliva collector cap 111 may include saliva deposition chamber 162, well 164, stem 110, and positive electrode 168 and negative electrode 170. Positive electrode 168 and negative electrode 170 are similar to positive electrode 138 and negative electrode 140, respectively. In one embodiment, saliva may be deposited in saliva collector cap 111 such that saliva makes contact with both electrodes 138 and 140 such that an electrical current may flow through the saliva, thus allowing conductance to be determined.

The above illustration provides many different embodiments or embodiments for implementing different features of the invention. Specific embodiments of components and processes are described to help clarify the invention. These are, of course, merely embodiments and are not intended to limit the invention from that described in the claims.

### Processing System

The methods and techniques described herein may be performed on a processor-based device. The processor-based device will generally comprise a processor attached to one or more memory devices or other tools for persisting data. These memory devices will be operable to provide machine-readable instructions to the processors and to store data. Certain embodiments may include data acquired from remote servers. The processor may also be coupled to various input/output (I/O) devices for receiving input from a user or another system and for providing an output to a user or another system. These I/O devices may include human interaction devices such as keyboards, touch screens, displays and terminals as well as remote connected computer systems, modems, radio transmitters and handheld personal communication devices such as cellular phones, “smart phones”, digital assistants and the like.

The processing system may also include mass storage devices such as disk drives and flash memory modules as well as connections through I/O devices to servers or remote processors containing additional storage devices and peripherals.

Certain embodiments may employ multiple servers and data storage devices thus allowing for operation in a cloud or for operations drawing from multiple data sources. The inventor contemplates that the methods disclosed herein will also operate over a network such as the Internet, and may be effectuated using combinations of several processing devices, memories and I/O. Moreover any device or system that operates to effectuate techniques according to the current disclosure may be considered a server for the purposes of this disclosure if the device or system operates to communicate all or a portion of the operations to another device.

The processing system may be a wireless device such as a smart phone, personal digital assistant (PDA), laptop, notebook and tablet computing devices operating through wireless networks. These wireless devices may include a processor, memory coupled to the processor, displays, keypads, WiFi, Bluetooth, GPS and other I/O functionality. Alternatively, the entire processing system may be self-contained on a single device.

The methods and techniques described herein may be performed on a processor-based device. The processor-based device will generally comprise a processor attached to one or more memory devices or other tools for persisting data. These memory devices will be operable to provide machine-readable instructions to the processors and to store data, including data acquired from remote servers. The processor will also be coupled to various input/output (I/O) devices for receiving input from a user or another system and for providing an output to a user or another system. These I/O devices include human interaction devices such as keyboards, touchscreens, displays, pocket pagers and terminals as well as remote connected computer systems, modems, radio transmitters and handheld personal communication devices such as cellular phones, “smart phones” and digital assistants.

The processing system may also include mass storage devices such as disk drives and flash memory modules as well as connections through I/O devices to servers containing additional storage devices and peripherals. Certain embodiments may employ multiple servers and data storage devices thus allowing for operation in a cloud or for operations drawing from multiple data sources. The inventors contemplate that the methods disclosed herein will operate over a network such as the Internet, and may be effectuated using combinations of several processing devices, memories and I/O.

The inventors further contemplate integration of embodiments of the present disclosure a network of nodes that are capable of performing some processing, gathering sensory information and communicating with other nodes in the network. Such wireless sensor nodes may include devices, vehicles, buildings and other items embedded with electronics, software, sensors, and network connectivity that enables the nodes to collect and exchange data (sometimes referred to as “Internet of Things” (IoT) or a wireless sensor network). In these embodiments, the inventors contemplate, by way of example and not limitation, a hydration sensor communicating with one or more hydration sensors and a base station. Said base station may coordinate data between the one or more hydration sensors. Administrators of the base station may use this data to inform the users to take or omit action, including but not limited to, hydrate. Further description of such embodiments are described herein.

The processing system may be a wireless device such as a smart phone, personal digital assistant (PDA), laptop, notebook and tablet computing devices operating through wireless networks. These wireless devices may include a processor, memory coupled to the processor, displays, keypads, WiFi, Bluetooth, GPS and other I/O functionality.

### FIG. 2

FIG. 2 illustrates a circuit diagram, according to one embodiment of the present disclosure. As shown, circuit 200 includes but is not limited to: oscillator 202, amplifier 204, AC/DC converter 205, microcontroller 206, wireless controller 124 (previously shown in FIG. 1B), battery 210, leads 212 (including poles alpha and beta), and antenna 122 (previously shown in FIG. 1B).

The role of oscillator 202 is to generate an alternating current in the form of a sine wave, however, those skilled in the art will appreciate that there are numerous other methods of generating an oscillating or sinusoidal current. By way of example and not limitation, oscillator 202 may take the form of a Wien bridge oscillator, as shown. While some embodiments, a single oscillating sine current may be used to take conductance measurements, other embodiments of the present disclosure use a broad frequency or multifrequency current (i.e., a combination of currents of varying frequency). In this manner, such currents may allow for saliva conductance measurements to be less affected by frequency-dependent confounders.

The role of amplifier 204 is to amplify the conductance of tested saliva.

However, those skilled in the art will appreciate that there are numerous other methods of amplifying an electrical signal. By way of example and not limitation, amplifier 204 may take the form of an operational amplifier.

Resistor R₁₀ represents saliva resistance. Poles alpha and beta represent the electrical connection to positive and negative conductance electrodes (such as positive electrode 138, negative electrode 140, and other electrodes described herein). In some embodiments, these electrodes assess the conductance of saliva to be tested. It is known in the arts that, as the conductance changes, resistance changes proportionately. Thus, in combination with amplifier 204 as pictured, amplification changes according to equation B.01, below:

A_{B}=R₈/R₁₀+1 Eq 2.1

Variables for Eq 2.1 are provided in Table 2.1, below:

TABLE 2.1

| | |
|---|---|
| A_{B} | Gain of Amplifier 204 |
| R₈ | Resistance of R₈ |
| R₁₀ | Resistance of R₁₀ |

Thus, saliva conductance is proportional to amplifier gain from amplifier 204.

In terms of enhancing saliva conductance measurements, it is important to take into account the following factors: electrode material and resistivity, electrode spacing (e.g., spacing between poles α and β ) as well as electrode surface contact area. The measured conductance is inversely related to the distance between the electrodes. Specifically, the conductance of saliva in siemens can be determined using Equation 2.2:

G=A_{S}/(ρL) Eq 2.2

The variables used in Equation 2.2 are provided in Table 2.2:

TABLE 2.2

| | |
|---|---|
| G | Conductance (e.g., of saliva) |
| A_{S} | Electrode surface area |
| ρ | Resistivity of saliva |
| L | Electrode spacing |

By way of example and not limitation, an electrode spacing of 7 mm, surface area of 4.15 mm², and the use of carbon electrodes may be successful in saliva conductance measurements.

The role of AC/DC converter 205, is to convert an alternating current (AC) into a direct current (DC) acceptable to a microcontroller or the like. However, those skilled in the art will appreciate that there are various methods of converting AC to DC. By way of example and not limitation, AC/DC converter 205 may take the form of a full-wave diode bridge rectifier, as shown.

The role of microcontroller 206 is to process the DC signal from AC/DC converter 205 into an output useful to the user, among other functions. However, those skilled in the art will appreciate that there are various methods of processing data. In one embodiment, microcontroller 206 outputs hydration levels based on the DC signal from AC/DC converter 205. In other embodiments, microcontroller 206 transmits hydration level data to the user via wireless controller 124, described below.

The role of wireless controller 124, is to propagate a wireless signal through spacetime to other devices. However, those skilled in the art will appreciate that there are variegated methods of transmitting a signal at different frequencies at different effective distances. By way of example and not limitation, wireless controller 124 may take the form of a combined bluetooth circuit and antenna. The role of antenna 122, is to generate an alternating current in the form of a sine wave, however, those skilled in the art will appreciate that there are numerous other methods of generating an oscillating or sinusoidal current.

Battery 210 provides power to circuit 200 such that circuit 200 is operational to perform saliva conductance testing. Those skilled in the art will recognize that there are numerous means of powering circuits, including electrical outlet power, inductive power, solar power and the like. Furthermore, those skilled in the art will recognize that circuits such as 200 will need a minimum of sustained and continuous power to operate, based on the load that circuit 200 causes on the power source.

Leads 212 may include poles alpha (α) and beta (β). In one embodiment, one or more of leads 212 are connected to a saliva conductance testing device (not pictured), such as saliva conductance tester 112.

By way of example and not limitation, suppose oscillator 202 has been configured to output an alternating current in the form of a sinusoidal wave with an amplitude of one volt (1V). Furthering the example, resistors are selected such that R8 is 2 kiloohms (kΩ). In keeping with this example, resistor R10 may represent the resistance (inverse conductance) of a user's saliva, which may have a resistance of 1 kΩ. If one uses a negative feedback amplifier as amplifier 204, the ratio of the amplitude of the output wave to the amplitude of the input wave from the oscillator will be governed by Equation B.1 above. The gain provided by such an amplifier is shown in Equation 2.3, below.

A_{B}=(2 kΩ/1 kΩ)+1=3. Eq. 2.3

(64) Thus, the gain provided by A_{B} may be 3, and therefore the output wave from the amplifier may constitute a 3V peak-to-peak sinusoidal wave, in keeping with the above example. Those skilled in the art will understand that converting an alternative current to a direct current may be a process that is less than perfectly efficient. In the exemplary full wave rectifier provided in circuit 200 (namely, AC/DC converter 205) a voltage drop may occur due to the diodes constituting the full-wave rectifier bridge of AC/DC converter 205. Furthering the example above, after passing through the AC/DC converter 205, the output may be different and varies based on the configuration of the rectifier. It is worth pointing out in the example that, depending on the resistance (conductance) of the saliva (in this example, R10), this DC signal may change in voltage, as affected by amplifier 204.

Continuing the example above, this DC signal may then be fed into, for example, a set of LEDs (not pictured). Those skilled in the art will understand that an increase in voltage may be used to power additional components, such as LEDs. In this example, additional LEDs may be lit based on voltage levels. Thus, one LED may be lit for voltages under 1V, a second LED may be lit for voltages under 2V, and the like. In this manner, saliva conductance may be indicated to the user through a selection of a number or color of LEDs. Those skilled in the art will understand that LEDs are merely one method of indicating a voltage output, a digital or analog readout, ammeter, voice callout, color change, screen display, and the like, and any and all means known in the art for representing or displaying information such as conductance and the like are contemplated.

Thus, one embodiment could be described as follows: two or more electrodes as described herein, a means of measuring saliva conductance; non-transitory memory, a power source and a processor.

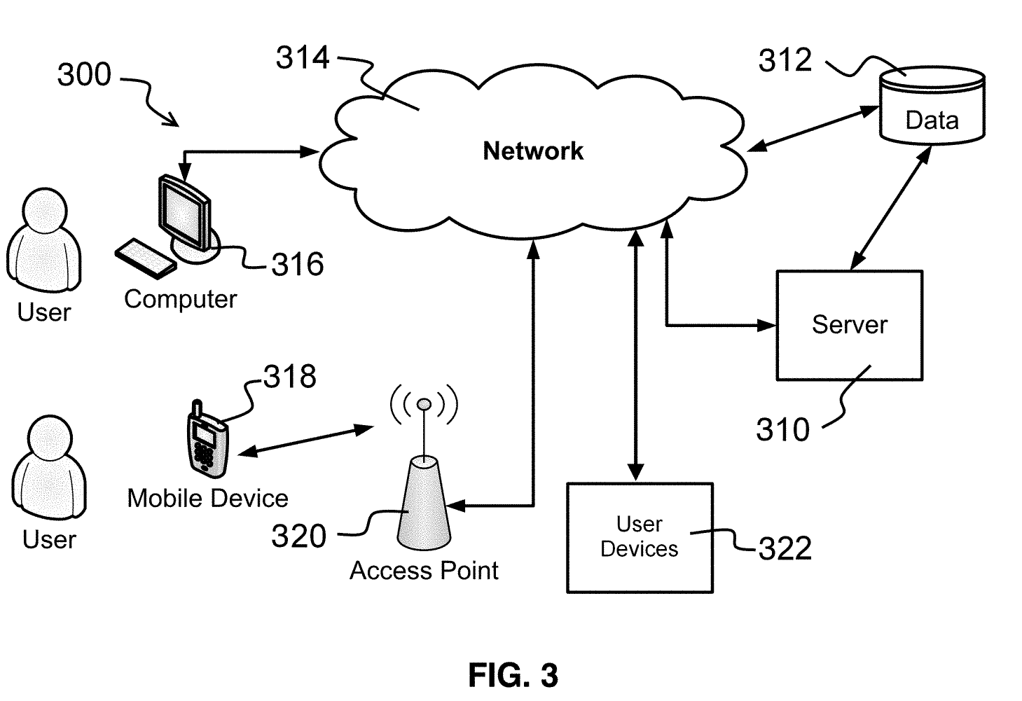

### FIG. 3

FIG. 3 illustrates a cloud computing environment, according to one embodiment of the present disclosure. As shown, FIG. 3 illustrates a functional block diagram of a client server system 300 that may be employed for some embodiments according to the current disclosure. In FIG. 3, a server 310 is coupled to one or more databases 312 and to a network 314. The network may include routers, hubs and other equipment to effectuate communications between all associated devices. A user accesses the server by a computer 316 communicably coupled to the network 314. The computer 316 includes a sound capture device such as a microphone (not shown). Alternatively the user may access the server 310 through the network 314 by using a smart device such as a telephone or PDA (mobile device) 318. Mobile device 318 may connect to the server 310 through an access point 320 coupled to the network 314. Mobile device 318 includes a sound capture device such as a microphone.

Conventionally, client server processing operates by dividing the processing between two devices such as a server and a smart device such as a cell phone or other computing device. The workload is divided between the servers and the clients according to a predetermined specification. For example in a “light client” application, the server does most of the data processing and the client does a minimal amount of processing, often merely displaying the result of processing performed on a server.

According to the current disclosure, client-server applications are structured so that the server provides machine-readable instructions to the client device and the client device executes those instructions. The interaction between the server and client indicates which instructions are transmitted and executed. In addition, the client may, at times, provide for machine readable instructions to the server, which in turn executes them. Several forms of machine readable instructions are conventionally known including applets and are written in a variety of languages including Java and JavaScript.

Client-server applications also provide for software as a service (SaaS) applications where the server provides software to the client on an as needed basis.

In addition to the transmission of instructions, client-server applications also include transmission of data between the client and server. Often this entails data stored on the client to be transmitted to the server for processing. The resulting data is then transmitted back to the client for display or further processing.

One having skill in the art will recognize that client devices may be communicably coupled to a variety of other devices and systems such that the client receives data directly and operates on that data before transmitting it to other devices or servers. Thus data to the client device may come from input data from a user, from a memory on the device, from an external memory device coupled to the device, from a radio receiver coupled to the device or from a transducer coupled to the device. The radio may be part of a wireless communications system such as a Wi-Fi or Bluetooth receiver. Transducers may be any of a number of devices or instruments such as thermometers, pedometers, health measuring devices and the like.

A client-server system may rely on “engines” which include processor-readable instructions (or code) to effectuate different elements of a design. Each engine may be responsible for differing operations and may reside in whole or in part on a client, server or other device. As disclosed herein a display engine, a data engine, an execution engine, a user interface (UI) engine and the like may be employed. These engines may seek and gather information about events from remote data sources.

In some embodiments according to the present disclosure, hydration sensors discussed herein may communicate with elements of network 314 through antenna 122 and wireless controller 124, or the like. In some embodiments, hydration sensors discussed herein may communicate with mobile device 318 through a bluetooth connection or other means known in the art. The contents of any communications may include but are not limited to: information relating to hydration levels of the user, water storage locations, water intake recommendations and the like, according to embodiments according to the present disclosure. In this manner, users of hydration sensors discussed herein and system administrators overseeing network 314 may make better-informed decisions relating to user hydration. More specific examples of enhanced decision making based on hydration levels and cloud-based communications

For example, a military setting may require strategic rationing and location of water, and such activities may be optimized through the use of embodiments according to the present disclosure, such as hydration sensors discussed herein. Specifically, decision makers of military supply logistics could deliver or relocate supplies based on water consumption/hydration levels by members of the military, as water is consumed. Furthermore, future such deliveries/relocations could be based on current consumption and hydration data recoded by embodiments according to the present disclosure in an effort to prevent supply shortages in the field. This future logistical planning may be easier to execute as locations vary in hostility. For example, if hydration sensors described herein record user water consumption as higher than current water storages accommodate for in a currently low-risk area, decision-makers may react accordingly by delivering more water while said areas are still low-risk. This is especially important if said areas are expected to become high-risk to members of the military. Such future logistical planning can be done in anticipation of these areas becoming actively hostile, making for safer and more effective supply delivery.

In another example, doctors, patients and researchers alike may benefit from hydration level monitoring and hydration level data sharing. Specifically, embodiments according to the present disclosure such as hydration sensors discussed herein may allow doctors to assess the hydration level of their patients as part of a standard vital sign check. Furthermore, the speed and portability of embodiments according to the present disclosure allow for unobtrusive and repeated usage of hydration sensors discussed herein such that hydration level monitoring, including real-time cloud storage of such data, may be done throughout the patient's stay. In keeping with the example, such cloud-stored hydration level data between one or more patients may be helpful for doctors to determine whether patients are properly hydrated, whether hydration levels are indicative of symptoms of unknown afflictions (thus potentially enhancing a physician's diagnosis), and the like. Researchers may benefit as well, in determining whether and how hydration levels affect various afflictions and patients alike, thus potentially enhancing clinical trial data. In addition, embodiments according to the present disclosure may assist pharmaceutical companies in determining the effects of hydration levels on the efficacy of medical drug therapy on patients.

In another example, embodiments according to the present disclosure could be used to naturally and safely enhance athlete performance through hydration level monitoring. For example, a water cooler or other hydration unit may include embodiments according to the present disclosure, such as hydration sensors discussed herein. Thus, when players hydrate, their hydration levels can be monitored and stored in the cloud. In this manner, player hydration levels can be tracked and allow for enhanced decision making by coaches, athletic trainers, and the like.

In another example, athletes or outdoor activity enthusiasts may carry embodiments according to the present disclosure such as hydration sensors discussed herein and record and trade hydration level data through social media, similar to the manner in which wearable device users do. In this manner, wearable device manufacturers, sports drinks manufactures and the like may use embodiments of the present disclosure to encourage more hydration and share their products enhanced with embodiments according to the present disclosure.

Thus, one system embodiment could be described as follows: a wireless network of hydration sensors, in which the hydration sensors include two or more electrodes, a means of measuring saliva conductance, a processor, non-transitory storage containing processor-based instructions, and a means of wireless communications, in which the non-transitory storage contains processor-based instructions operable for determining hydration levels, in which the hydration sensors are operable to output hydration status data; and a base station, in which the base station coordinates hydration status data between the hydration sensors.

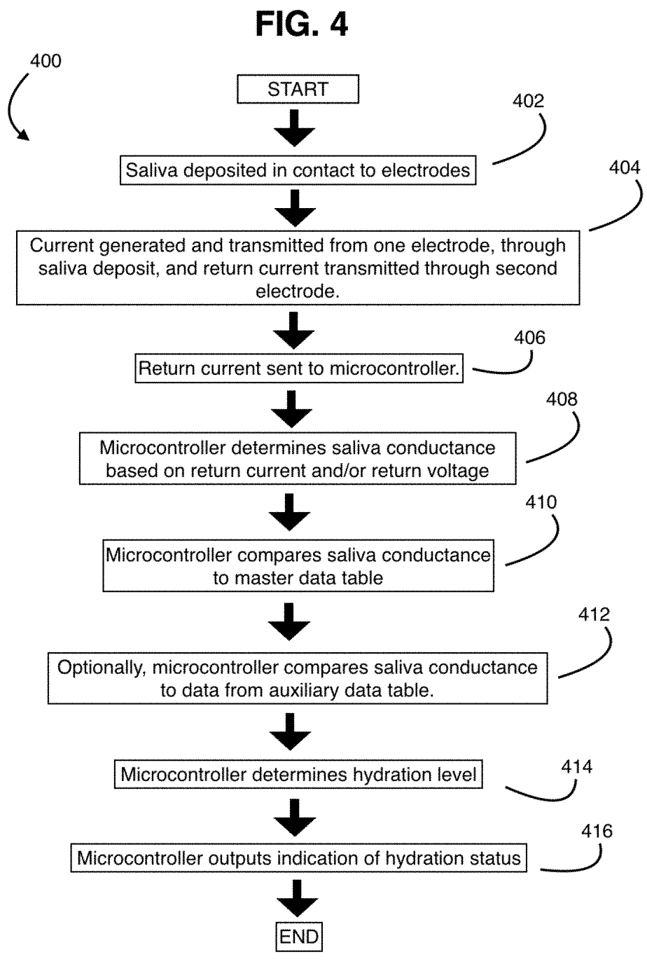

### FIG. 4

FIG. 4 illustrates a flow diagram of method steps for determining hydration levels, according to one embodiment of the present disclosure. Although the method steps are described in conjunction with FIGS. 1-10, persons skilled in the art will understand that any system configured to perform the method steps, in any order, falls within the scope of the present invention. The steps in this method are illustrative only and do not necessary need to be performed in the given order they are presented herein. Some steps may be omitted completely.

The method begins at a step 402, a user depositing a sample of the user's saliva in contact to electrodes described herein, such electrodes may be part of saliva conductance tester 112 or catheters described herein.

At a step 404, a current is generated (in some embodiments, by oscillator 202) and sent to electrodes described herein. In some embodiments, this current is an alternating current or a multifrequency current. In this step, the generated current is transmitted from one electrode, through the saliva deposited, and collected by the second electrode.

At a step 406, the return current is accepted by a microcontroller or the like.

In this step, a return current may be sent to an AC to DC converter. In some embodiments, this return current is an alternating current. In these embodiments, the return current is converted into a direct current.

At a step 408, the microcontroller determines saliva conductance based on the return current and/or return voltage.

At a step 410, the microcontroller compares saliva conductance to a master data table. The master data table may contain but is not limited to one or more of the following: saliva conductances and hydration levels.

At an optional step 412, the microcontroller compares saliva conductances to data from an auxiliary data table. The auxiliary data table may contain but is not limited to one or more of the following: hydration levels, osmolarity, osmolality, ion concentration, body weight, body weight differential data due to water intake, gender, age, ethnicity, medical history data, fitness levels (such as body mass index and the like), urine data, and blood data. In some embodiments, data from auxiliary data table may be reflective of the user.

At a step 414, the microcontroller determines hydration level.

At a step 416, the microcontroller outputs an indication of the hydration status, after which the method ends.

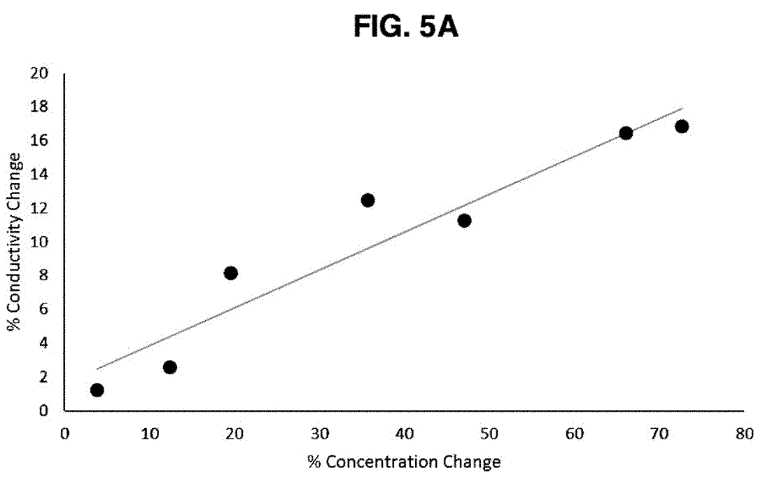

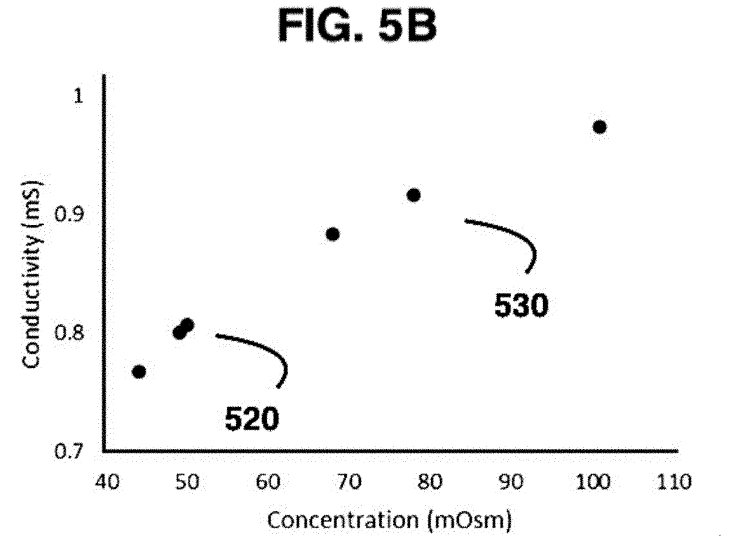

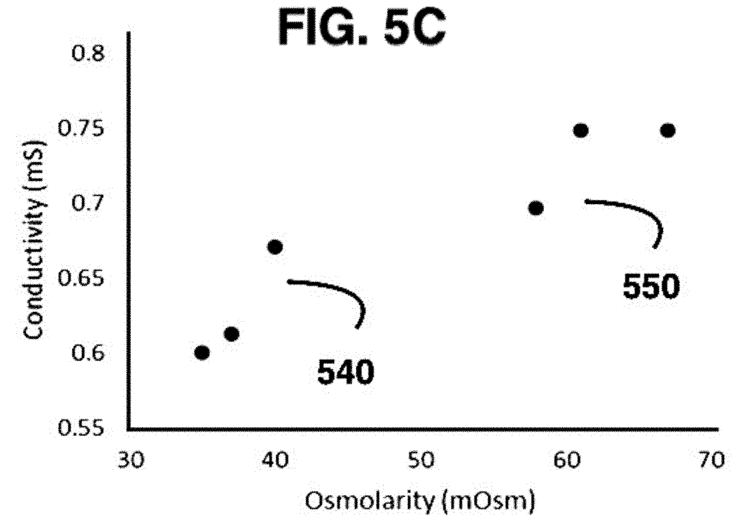

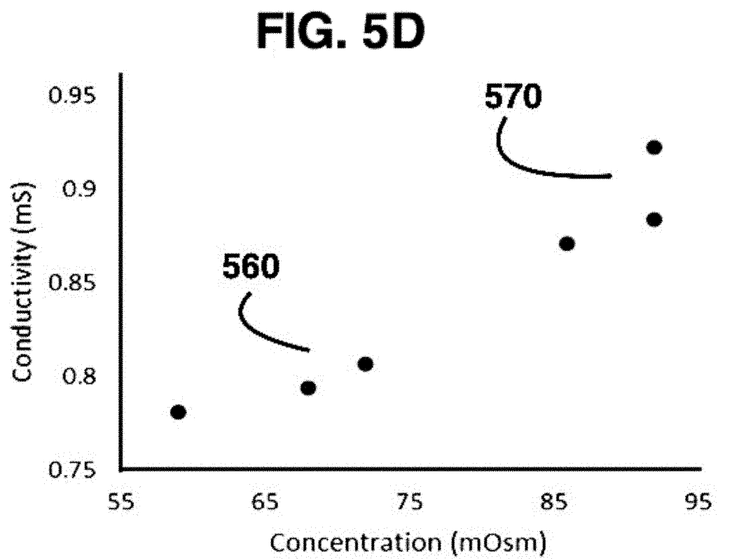

### FIG. 5

FIG. 5 illustrates graphs indicating saliva conductance vs. osmotic concentration in human subjects, according to embodiments of the present disclosure. More specifically, Graph 5A shows a linear regression plot of empirical data taken from the saliva of seven human test subjects. Discussed herein are various methods of measuring human hydration levels, followed by a discussion of interpreting saliva conductance data as a means of determining hydration level, according to embodiments according to the present disclosure.

Using ionic concentration as a means of determining hydration status will be discussed. Those skilled in the art know that saliva consists of, among other things, ions and biological components (e.g., mucin). Measuring the concentration of ions in saliva from an individual may provide a determination of the individual's hydration status. However, measuring ionic concentration directly can be cumbersome, time-consuming and difficult, especially in outdoor or during vigorous activity conditions. As conventionally known, an ion selective electrode may be used to test for ionic concentration, but such a device may require tuning and individual measurements for each type of ion to be measured. This is impractical for use during in-field saliva ion measurement due to the fact that saliva contains numerous, variegated ion types. Furthermore, a ion selective electrode is a bench-top measurement system best suited for laboratory use, rather than in-field (e.g., military field or outdoor) use.

According to embodiments according to the present disclosure, conductance may be an indirect measure of a solution's osmolarity, and conductance may be a sensing modality that can be much more easily translated into a portable device.

Discussed herein is the correlation between saliva conductance and hydration. A dehydrated subject is likely to produce saliva that contains less water (dehydrated saliva) than saliva from a hydrated subject (hydrated saliva). This saliva is likely to be higher in ion concentration with ions, and more conductive. Thus, a saliva sample's ion concentration may be electrically determined through conductance. More specifically, a percent change in conductance relative to a hydrated state may be used to detect the onset of dehydration.

Using conductance as an indirect measure of osmolarity is possible due to the correlation between these two metrics, as shown in Graph 5A, which shows a linear regression plot of conductance measurements versus osmolarity. More specifically, Graph 5A illustrates seven data points that each correspond to two human saliva samples taken from individuals. One sample was taken prior to exercise, and a second sample taken after exercise. In this study, exercise was 45 minutes of vigorous activity.

Each pair of saliva samples was tested for conductance and ion concentration. Then the conductance values post-exercise were subtracted from the conductance values pre-exercise for each pair of saliva samples. Similarly, the ion concentration values post-exercise were subtracted from the conductance values pre-exercise for each pair of saliva samples. FIG. 5A shows the percent change for ion concentration versus conductance changes. Importantly, a clear trend emerged, with the square of a correlation coefficient R² of 0.9159. Based on this correlation coefficient, changes in the Y values (conductance) can be partly attributed to the changes in the X values (ion concentration) for each pair of saliva samples, pre- and post-exercise. It is important to note that a correlation coefficient value of 0.9159 clearly indicates a trend, considering the degree of variability in contents and quality of actual biological saliva samples and error that may occur in empirical testing generally.

Since conductance is closely related to osmolarity (as described above), conductance of saliva may be used as an approximation of saliva's osmolarity, according to embodiments according to the present disclosure. In this manner, osmolality, and thus user hydration level may be approximated through conductance measurements of the user's saliva.

FIG. 5 also illustrates Graphs 5B, 5C and 5D, each indicating saliva conductance vs. saliva osmotic concentration pre- and post-exercise in three human subjects, one subject for each of the Graphs 5B-D. As shown, Graphs 5B-D reflect the results of an experimental trial in which volunteer subjects were asked to dehydrate themselves by exercising without drinking water. Saliva samples were collected both before exercising and after exercising, and subject body weight was also measured before and after exercise to determine the percent change in body weight due to water loss.

Pre-workout hydration results for the three subjects are shown in Groupings 520, 540 and 560, respectively. Post-workout hydration results for the three subjects are shown in Groupings 530, 550, 570, respectively. A distinct clustering can be seen between pre-workout and post-workout samples. These clusterings evidence the same trend of correlated saliva osmolarity to subject dehydration, further bolstering the concept that user hydration level may be approximated through conductance measurements of the user's saliva.

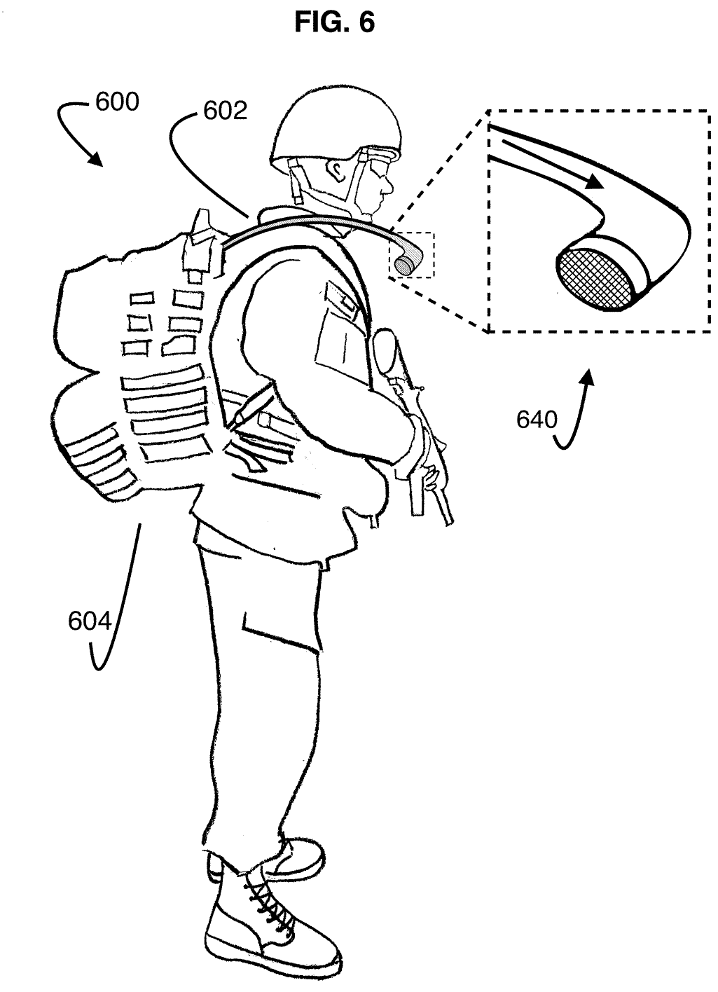

### FIG. 6

FIG. 6 illustrates a hydration sensor integrated into military equipment, according to one embodiment of the present disclosure. As shown, 600 includes catheter 602 and backpack 604. In some embodiments catheter 602 may be a standard hydration catheter known in the art, and backpack 604 may contain a separate compartment for a removable, handheld hydration sensors as discussed herein. In this manner, a user may check their hydration levels as needed. In further embodiments, hydration level data may be wirelessly uploaded into the cloud or act as a node in a wireless sensor network or IoT network, as described herein.

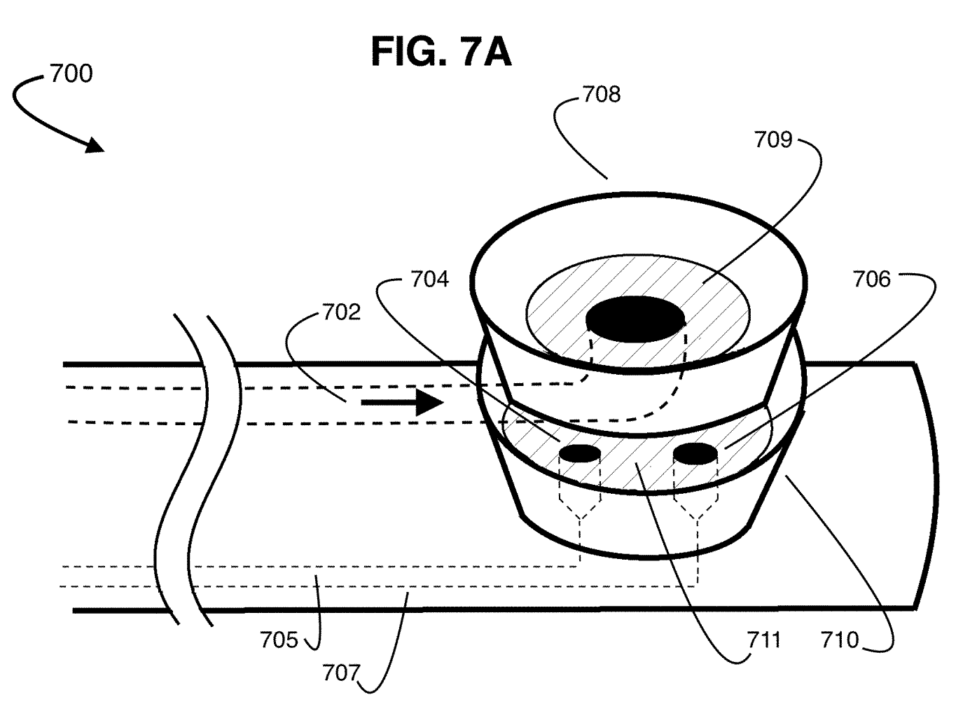

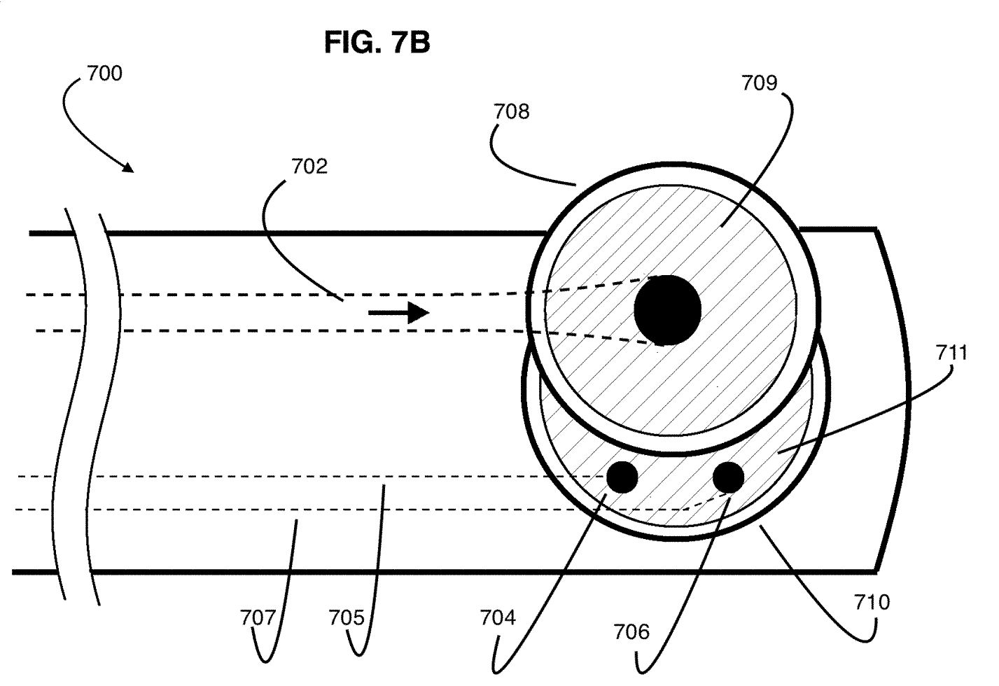

Catheter 602 allows for fluid flow, as shown by the arrow in inset 640. In some embodiments, catheter 602 may be multifunction catheter 700, multifunction catheter 800, multifunction catheter 902, or any hydration catheter described herein. It should be noted that FIG. 6 is meant to illustrate exemplary uses of hydration sensors discussed herein, and should not be read as limiting; thus other embodiments described in this patent application not pictured here may be used in military and other settings. FIG. 7

FIGS. 7A and 7B illustrate an integrated hydration sensor and hydration catheter, according to embodiments according to the present disclosure. As shown, FIG. 7A includes multifunction catheter 700, which houses a hydration tube 702, and positive electrode 704 and negative electrode 706, and optionally, data leads 705 (not pictured). Hydration tube 702 allows fluid flow, as demonstrated by the arrow inside hydration tube 702. FIG. 7B illustrates multifunction catheter 700 from another view. The hydration catheter also includes hydration catheter well 708, and saliva deposition chamber 710. Finally, hydration catheter well 708 has hydration catheter well floor 709, hydration well opening 707, and saliva deposition chamber 710 has saliva deposition chamber floor 711. Optionally, multifunction catheter 700 may include a locking mechanism (not shown) and/or a flexible enclosure (not shown) to limit fluid discharge, as known in the art. In some embodiments, positive electrode 704 and negative electrode 706, and optional data leads 721 (not pictured) may be electrically connected to hydration sensors discussed herein, through electrical connections 705 and 707. In other embodiments, positive electrode 704, negative electrode 706 and other elements shown in the Figure may be structurally sound such that these elements are resistant to crushing forces (e.g., biting).

Conventionally, catheters may be attached to reservoirs stored in “hydration backpacks,” which are often used in athletic or extreme environments, including but not limited to, outdoor sports, (e.g., cycling, hiking or climbing) and military combat. In this manner, a hydration backpack user may sip water or other fluids stored in the reservoir contained in the hydration backpack. The user sips fluid by means of suction through multifunction catheter 700.

Saliva deposition chamber 710 is similar to saliva deposition chamber 162 in that saliva deposition chamber 710 may be used to test the conductance of saliva deposited in saliva deposition chamber 710, according to embodiments according to the present disclosure. Saliva deposition chamber 710 differs from saliva deposition chamber 162 in that saliva deposition chamber 710 is mounted in proximity to hydration catheter well 708. Positive electrode 704 and negative electrode 706 serve to test the conductance of saliva deposited. In this manner, a user may deposit saliva in saliva deposition chamber 710, which allows hydration sensors discussed herein to determine the conductance of the deposited saliva. In this manner, hydration sensors (as discussed herein) may avoid reading the conductance of other fluids, which may be beneficial to accuracy of saliva conductance measurements.

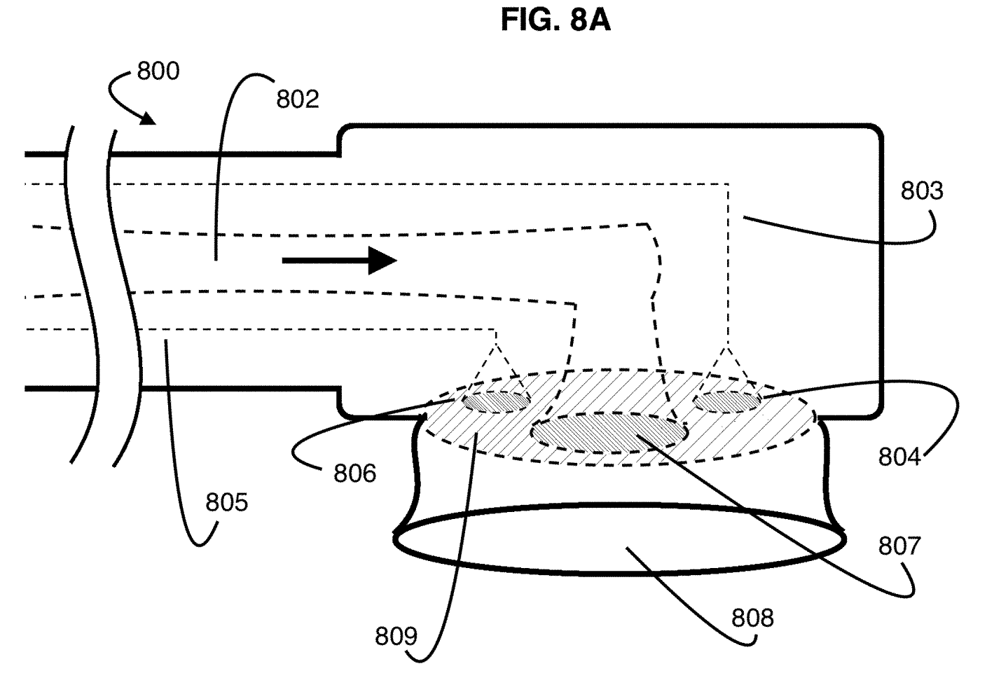

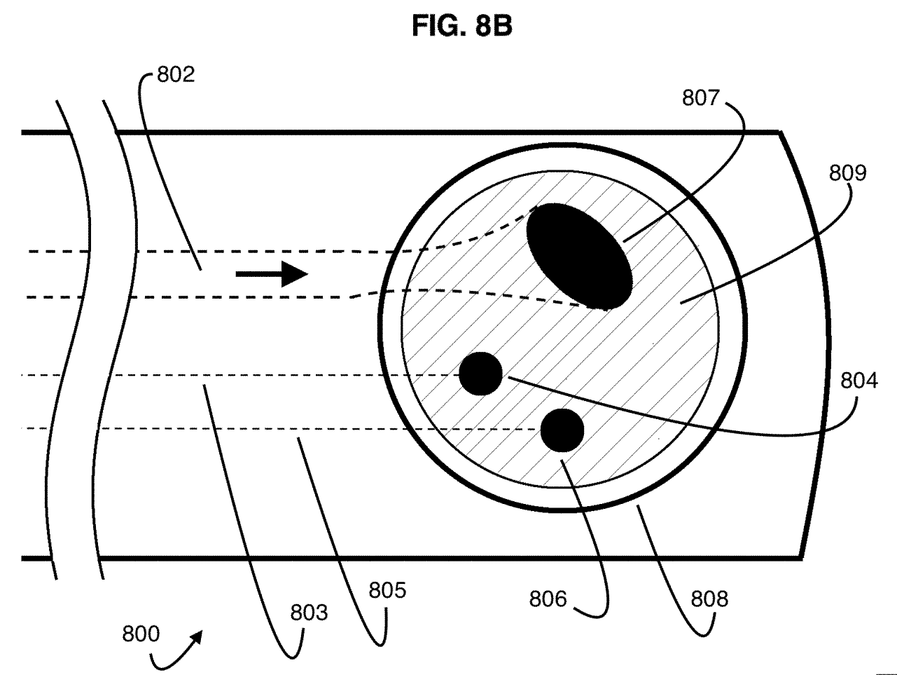

### FIG. 8

FIGS. 8A and 8B illustrate an integrated hydration sensor and hydration catheter, according to embodiments according to the present disclosure. As shown, FIG. 8A includes multifunction catheter 800, which houses a hydration tube 802, and positive electrode 804 and negative electrode 806, and optionally, data leads 821 (not shown). Hydration tube 802 allows for fluid flow, as demonstrated by the arrow inside hydration tube 802. Positive electrode 804 and negative electrode 806 may be electively connected to hydration sensors described herein through electrical connections 803 and 805. FIG. 8B illustrates multifunction catheter 800 from another view. Multifunction catheter 800 also includes integrated hydration/saliva well (multifunction well) 808. Multifunction well 808 has hydration well opening 807.

Optionally, multifunction catheter 800 may include a locking mechanism (not shown) and/or a flexible enclosure (not shown) to limit fluid discharge, as known in the art. In some embodiments, positive electrode 804 and negative electrode 806, and optional data leads 805 are electrically connected to hydration sensors discussed herein.

Multifunction well 808 is similar to saliva deposition chamber 162 in that saliva deposition chamber 810 may be used to test the conductance of saliva deposited in multifunction well 808. Positive electrode 804 and negative electrode 806 serve to test the conductance of saliva deposited. In this manner, a user may deposit saliva in multifunction well 808, which allows hydration sensors discussed herein to determine the conductance of the deposited saliva.

According to embodiments of the present disclosure, multifunction well 808 differs from saliva deposition chamber 162 in that multifunction well 808 serves as both a location for the user to deposit saliva for conductance testing by hydration sensors discussed herein, as well as a location for dispensing fluid through hydration tube 802. In some embodiments, positive electrode 804 and negative electrode 806 and hydration well opening 807 share the same “floor,” specifically, multifunction well floor 809. In other words, multifunction well floor 809 has hydration well opening 807 in proximity to positive electrode 804 and negative electrode 806.

In some embodiments, automatic testing of saliva may be performed by hydration sensors discussed herein before or after the user sips fluids through hydration catheter 800. Conveniently, in this manner, a user may not be required to separately test the user's saliva for dehydration; hydration levels may be automatically determined as part of the user's regular use of hydration catheter 800.

In further embodiments, hydration sensors discussed herein may detect that the user did not hydrate enough with a previous sip, and thus hydration sensors discussed herein may encourage the user to sip more fluids. Beneficially, in this mariner, injuries dehydration may be avoided or mitigated. Such encouragement may occur through through means of lit LEDs, voice reminders or other means known in the art.

In even further embodiments, hydration sensors discussed herein may detect that the user may have over-hydrated with a previous sip, and thus hydration sensors discussed herein may discourage the user from imbibing more fluids. Beneficially, in this manner, hydration sensors discussed herein may assist the user in rationing fluid use by conserving onboard fluids stored in reservoir (not pictured). Such discouragement may occur through means of lit LEDs, numerical displays, voice reminders or other means known in the art.

In general, hydration sensors discussed herein may be activated such that positive electrode 804 and negative electrode 806 do not test for conductance in the presence of fluid from the reservoir. Such fluid presence may negatively impact conductance testing, causing inaccurate conductance measurements. In other embodiments, positive electrode 804, negative electrode 806 and other elements in this Figure may be structurally sound such that these elements are resistant to crushing forces (e.g., biting).

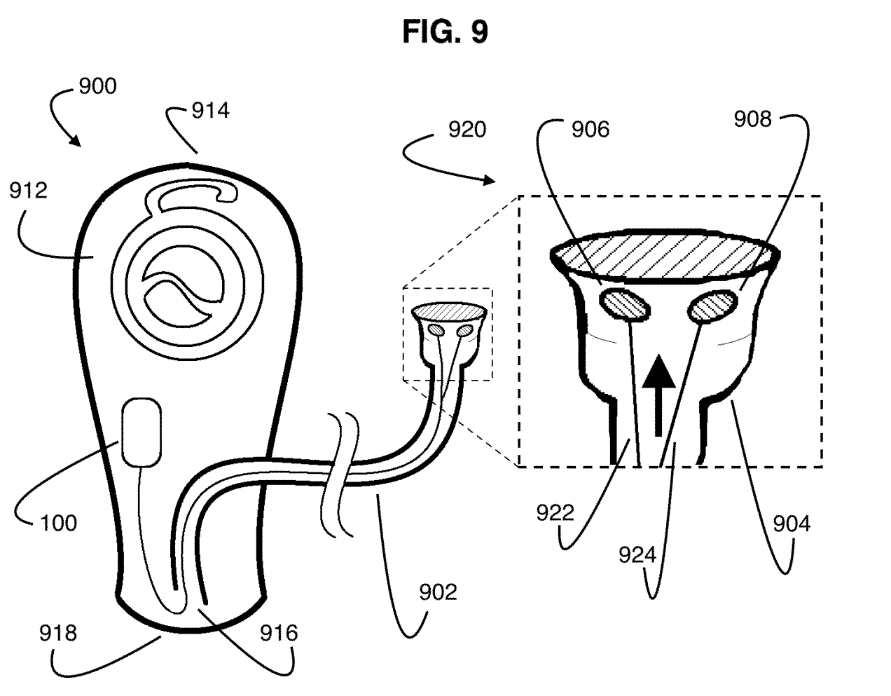

### FIG. 9

FIG. 9 illustrates an integrated hydration sensor and hydration catheter and reservoir, according to embodiments according to the present disclosure. As shown, FIG. 9 includes reservoir 900, a hydration sensor as discussed herein, (by way of example and not limitation, hydration sensor 100 is shown), integrated wire/catheter tube (multifunction catheter) 902, and integrated hydration/electrode mouthpiece (multifunction mouthpiece) 904. Reservoir 900 includes screw cap 912, hanger 914, catheter mount 916 and bladder 918.

By way of example and not limitation, hydration sensor 100 is attached to reservoir 900, but hydration sensor 100 may be located anywhere proximate to multifunction catheter 902. Fluid may flow through multifunction catheter 902, as indicated by the arrow within multifunction catheter 902.

As shown in inset 920, multifunction mouthpiece 904 includes positive electrode 906 and negative electrode 908, which may be electrically connected by electrical connections 922 and 924 to hydration sensors discussed herein, according to the present disclosure. In one embodiment, the user may partially or completely engulf multifunction mouthpiece 904 in saliva. In this manner, hydration sensors discussed herein may determine the conductance of the user's saliva.

In some embodiments, positive electrode 906, negative electrode 908 and a other elements in this Figure may be structurally sound such that these elements are resistant to crushing forces (e.g., biting).

In some embodiments, automatic testing of saliva may be performed by hydration sensors discussed herein before or after the user sips fluids through multifunction catheter 902. Conveniently, in this manner, a user may not be required to separately test the user's saliva for dehydration; hydration levels may be automatically determined as part of the user's regular use of multifunction catheter 902.

In further embodiments, hydration sensors discussed herein may detect that the user did not hydrate enough with a previous sip, and thus hydration sensors discussed herein may encourage the user to sip more fluids. Beneficially, in this mariner, injuries dehydration may be avoided or mitigated. Such encouragement may occur through means of lit LEDs, voice reminders or other means known in the art.

In even further embodiments, hydration sensors discussed herein may detect that the user may have over-hydrated with a previous sip, and thus hydration sensors discussed herein may discourage the user from imbibing more fluids. Beneficially, in this manner, hydration sensors discussed herein may assist the user in rationing fluid use by conserving onboard fluids stored in reservoir (not pictured). Such discouragement may occur through means of lit LEDs, voice reminders or other means known in the art.

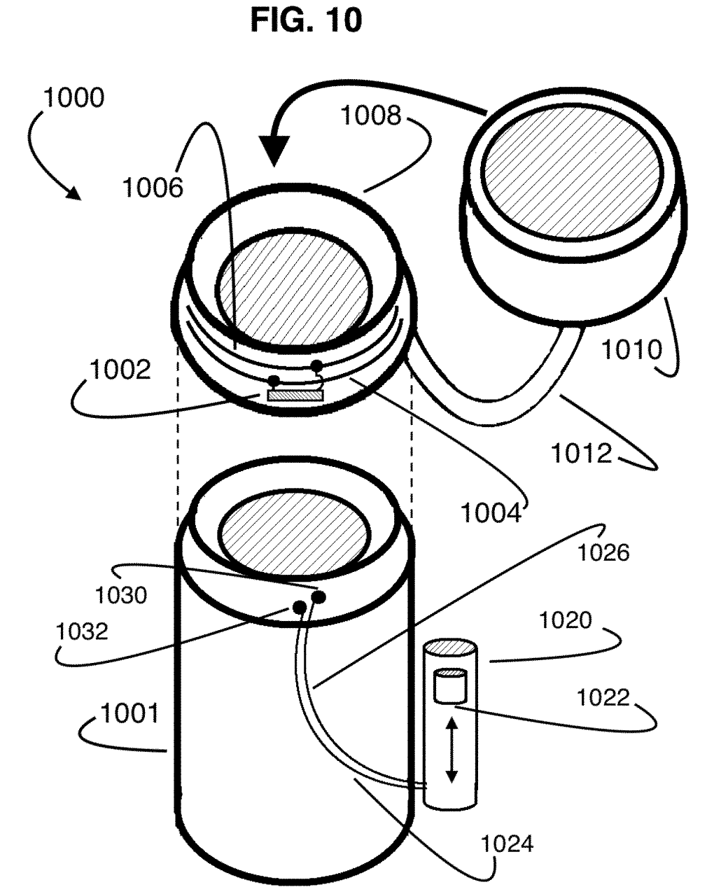

### FIG. 10

FIG. 10 illustrates a hydration sensor attached to a hydration bottle system, according to one embodiment of the present disclosure. As shown, hydration bottle system 1000 includes hydration sensor 1002, which may include positive electrode 1004 and negative electrode 1006. Hydration bottle system 1000 may include bottle 1001, smart lid anchor 1008 and lid 1010. Lid 1010 may be attached to smart lid anchor 1008 using strap 1012. Smart lid anchor 1008 may be attached to bottle 1001 by screwing, gluing or other attachment means known in the art. The inventors note that numerous permutations of hydration sensors 1002, bottles, lids, lid anchors and straps are possible, and the inventors contemplate any and all combinations of these elements. One skilled in the art will understand that embodiments of the invention are not limited to those illustrated herein.

In some embodiments, positive electrode 1004 and negative electrode 1006 are electrically connected to hydration sensor 1002. Hydration sensor 1002 may be substantially similar to hydration sensors discussed herein, such as hydration sensor 100. In some embodiments, hydration sensor 1002 is attached to smart lid anchor 1008 temporarily by the user by affixing, screwing or by any means of attachment known in the art. In other embodiments, hydration sensor 1002 may be secured by a manufacturer to smart lid anchor 1008 by gluing or other means of attachment known in the art. In some embodiments, hydration sensor 1002 may be a thin or printable circuit (e.g., RFID or the like) that is secured or printed directly onto hydration bottle 1001 or lid 1010 by means known in the art. In some of these embodiments, these means of securing hydration sensor 1002 to hydration are liquid-proof or otherwise resistant to liquids.

In some embodiments, hydration sensor 1002 may include batteries (not pictured, such as battery 210), may be powered by inductive charging through mutual inductance, or other means known in the art. We contemplate various charging systems, including a ‘chargeable hydration bottle’ such that a user may charge hydration sensor 1002 on in a charging station, or with a photovoltaic cell (not shown, e.g., a solar panel) or the like. Such a charging station may be combined with a hydration station.

Other charging systems include a shaker charger, as shown by optional shaker charger 1020. A shaker charger allows the user to shake ferrite core 1022 suspended in an inductive coil (not shown) in a relatively linear motion, such shaking then inducing a current appropriate for charging hydration sensor 1002 or hydration sensors discussed herein. Current induced by shaker charger 1020 may be fed through electrical connections 1024 and 1026 to contacts 1030 and 1032. Contacts 1030 and 1032 may be electrically connected to hydration sensor 1002 when smart lid anchor 1008 is affixed to bottle 1001. Such a shaker charger may be additionally beneficial in allowing simultaneous charging of hydration sensors 100 or hydration sensors 1100 as well as mixing, emulsifying or otherwise redistributing the contents of hydration bottle system 1000. For example, a user could shake hydration bottle system 1000, thereby charging hydration sensor 1002 and mixing a smoothie within hydration bottle system 1000.

In one embodiment, positive electrode 1004 and negative electrode 1006 may extend partially or completely around the circumference of the spout of smart lid anchor 1008. In this mariner, a user may drink from hydration bottle 1002 from multiple angles, and hydration sensors discussed herein may still receive hydration level data from the user's saliva. Thus, the user is less restricted when attempting to determine the user's hydration level.

The inventors also contemplate another embodiment: positive electrode 1004 and negative electrode 1006 may extend partially or completely around the circumference of the spout of hydration bottle 1002, omitting smart lid anchor 1008 entirely. In this manner, hydration bottle 1002 would include hydration sensor 1002 on the spout of hydration bottle 1002.

In a further embodiment, lid 1010 is optional and is not required for the user of hydration sensor 1002. In an even further embodiment, hydration sensor 1002 may be attached to contest 1030 and 1032. In this embodiment, hydration sensor 1002 may be powered by optional shaker charger 1020. In an alternative embodiment, smart lid anchor 1008 may contain a power source (not shown) and may function as a replacement lid anchor and optional lid. In this manner, the user may use smart lid anchor 1008 with conventional hydration bottles by simply attaching the threading of smart lid anchor 1008 to the conventional hydration bottle (by means of screwing or the like).

Although the invention is illustrated and described herein as embodied in one or more specific examples, it is nevertheless not intended to be limited to the details shown, since various modifications and structural changes may be made therein without departing from the spirit of the invention and within the scope and range of equivalents of the claims. Accordingly, it is appropriate that the appended claims be construed broadly and in a manner consistent with the scope of the invention, as set forth in the following claims.

We claim:

1. A system comprising:
a network comprising at least one hydration sensor,
wherein the at least one hydration sensor includes two or more electrodes, a means of measuring saliva conductance, a processor, non-transitory storage containing processor-based instructions, and a means of communications,
wherein the non-transitory storage contains processor-based instructions operable for determining hydration levels,
wherein the at least one hydration sensor is operable to output hydration status data; and
at least one base station,
wherein the at least one base station coordinates hydration status data between the at least one hydration sensor.

2. The system of claim 1
wherein the hydration status data indicates dehydration,
wherein the hydration status data operates the at least one base station to indicate to user of the at least one hydration sensor to hydrate.

3. The system of claim 1
wherein the hydration status data indicates normal hydration or over-hydration,
wherein the hydration status data operates the at least one base station to indicate to user of the one of the at least one hydration sensor to cease hydrating.

4. A processor-based method operable for measuring the conductance of saliva, the method comprising the steps of:
depositing saliva on two or more electrodes;
passing a current through the saliva deposit and the electrodes;
determining the conductance of the saliva using a processor; and
determining a hydration level using a processor.

5. The method of claim 4, further comprising the steps of:
receiving a hydration level indicating dehydration; and
displaying an indication to hydrate.

6. The method of claim 4, further comprising the steps of:
receiving a hydration level indicating normal hydration status or over-hydration status; and
displaying an indication to cease hydrating.

7. The method of claim 4, further comprising the steps of:
comparing saliva conductance to a master data table using a processor,
wherein the master data table contains one or more of the following:
saliva conductance data or hydration level data.

8. The method of claim 4, further comprising the steps of:
comparing saliva conductance data to data from an auxiliary data table using a processor,
wherein the auxiliary data table contains one or more of the following:
hydration level data, osmolarity data, osmolality data, ion concentration data, body weight data, body weight water loss differential data, gender data, age data, ethnicity data, medical history data, fitness level data, urine data, and blood data.

9. A processor-based apparatus for determining hydration levels in humans, the apparatus comprising:
two or more electrodes;
a circuit operable to measure the conductance of bodily fluids;
non-transitory memory;
a power source; and,
a processor.

10. The apparatus of claim 9, further comprising:
a hydration catheter,
wherein the hydration catheter includes a hydration catheter well and a hydration opening.

11. The apparatus of claim 10, wherein the two or more electrodes are proximate to the hydration catheter well.

12. The apparatus of claim 10, further comprising:
a saliva deposition well proximate to the hydration catheter well,
wherein the two or more electrodes are proximate to the saliva deposition well.

13. The apparatus of claim 10, further comprising:
a hydration reservoir attached to the hydration catheter,
wherein the hydration reservoir is operable to allow fluid flow from the hydration reservoir through the hydration catheter and through the hydration opening.

14. The apparatus of claim 9, further comprising:
a bottle; and
a lid,
wherein the two or more electrodes are disposed on the bottle.

15. The apparatus of claim 14, wherein the power source is an inductive charger.

16. The apparatus of claim 14, wherein the power source is a solar cell.

17. The apparatus of claim 9, wherein the electrodes are composed of at least one of: carbon, gold, platinum, copper, or a conductive polymer.

18. The apparatus of claim 9, wherein the electrodes are resistant to crushing.

19. The apparatus of claim 9, further comprising:
a display means, wherein the display means is operable to display a hydration status.

20. The apparatus of claim 9, further comprising:
a wireless communication means, wherein the wireless communication means is operable to share a hydration status on the Internet.
